(19) United States
(12) Patent Application Publication
(10) Pub. No.: US 2022/0040302 A1
(43) Pub. Date: Feb. 10, 2022

(54) Method and apparatus for producing non-local physical, chemical and biological effects

(71) Applicant: Huping Hu, Stony Brook, NY (US)

(72) Inventor: Huping Hu, Stony Brook, NY (US)

(21) Appl. No.: 17/493,081

(22) Filed: Oct. 4, 2021

Related U.S. Application Data
(63) Continuation-in-part of application No. 11/944,631, filed on Nov. 25, 2007.

(51) Int. Cl. A61K 41/00

(52) U.S. Cl. CPC A61K 41/0004

(57) ABSTRACT

A method and apparatus are disclosed which produce physical, chemical and/or biological non-local effect on a target substance through non-local processes mediated by quantum entanglement. In one broad embodiment, the apparatus includes a target substance, a first container holding said target substance; an originating substance, a second container holding said originating substance, said originating substance being quantum-entangled with said target substance; and a mean for manipulating said originating substance such that when said manipulation mean operates, said non-local effect is generated in said target substance through said non-local processes mediated by quantum entanglement. Also described are a number of implementations and methods of use of the apparatus, including a member of the manipulation mean being a Dewar filled with liquid nitrogen, a heater, a chemical substance, a laser or a magnetic coil connected to a driving device and the use being industrial, therapeutic, communicational or recreational.

### CROSS-REFERENCE TO RELATED APPLICATIONS

This application is a continuation-in-part application of U.S. patent application Ser. No. 11/944,631 filed on Nov. 25, 2007, which claims priority from U.S. provisional application Ser. No. 60/869,511 filed Dec. 11, 2006, which applications are fully incorporated herein by reference.

### FIELD OF THE INVENTION

The invention herein relates to method of producing non-local physical, chemical and biological effects on physical, chemical and/or biological systems through quantum entanglement mediated processes, to apparatus for such productions, and to method of using the non-local effects for beneficial purposes.

### BACKGROUND OF THE INVENTION

Many experiments have shown that quantum entanglement is physically real (see Aspect, A., Dalibard, J., & Roger, G. Experimental test of Bell's inequalities using time-varying analyzers. Phys. Rev. Lett. 49, 1804-1807 (1 982)). It is ubiquitous in the microscopic world and manifests itself macroscopically under some circumstances (see Ghosh, S., Rosenbaum, T. F., Aeppli, G. & Coppersmith, S. N. Entangled quantum state of magnetic dipoles. Nature 425, 48-51 (2003)). Indeed, quantum spins of photons, electrons and nuclei have now been successfully entangled in various ways for purposes of quantum computation and communication (see Matsukevich, D. N. & Kuzmich, A. Quantum state transfer between matter and light. Science 306, 663-666 (2004)).

However, the essence and implications of quantum entanglement are still hotly debated and largely unknown. For example, it is commonly believed that quantum entanglement alone cannot be used to transmit binary or classical information. Further, despite of the fact that all interactions in biological systems at molecular and sub-molecular levels are quantum interactions in nature, it is commonly believed that quantum effects do not play any roles in biological functions such as brain functions due to quantum decoherence (see Tegmark, M. The importance of quantum decoherence in brain processes. Phys. Rev., 61 E: 4194 (2000)). Yet, I have recently discovered non-local effects of chemical substances on biological systems such as a human brain produced through quantum entanglement (Hu, H. P., & Wu, M. X. Photon induced non-local effect of general anesthetics on the brain. NeuroQuantology 4,17-31 (2006); Hu, H. P., & Wu, M. X. Non-local effects of chemical substances on the brain produced through quantum entanglement. Progress in Physics v3, 20-26 (2006)).

My invention and discovery were made against such background. No process has previously been known which can produce non-local physical, chemical, thermal and gravitational effects through quantum entanglement mediated processes on targets such as physical, chemical or biological systems, so that beneficial effects or information can be delivered through said processes.

### SUMMARY OF THE INVENTION

I have now invented apparatus and method for producing non-local physical, chemical, thermal and gravitational effects through quantum entanglement mediated processes on targets such as physical, chemical or biological systems.

The subject invention is originated from my research on brain functions and nature of quantum entanglement. I have theorized that nuclear and/or electronic spins inside brain play important roles in certain aspects of brain functions such as perception (Hu, H. P., & Wu, M. X. Spin-mediated consciousness theory. Medical Hypotheses 63, 633-646 (2004); also see arXiv e-print quant-ph/0208068v1 (2002)). Further, I have discovered non-local effects of chemical substances on biological systems such as a human brain produced through quantum entanglement (Hu, H. P., & Wu, M. X. Photon induced non-local effect of general anesthetics on the brain. NeuroQuantology 4, 17-31 (2006); Hu, H. P., & Wu, M. X. Non-local effects of chemical substances on the brain produced through quantum entanglement. Progress in Physics v3, 20-26 (2006)).

The subject invention is therefore based on my realizations that (1) quantum entanglement means genuine interconnectedness and inseparableness of once interacting quantum entities and can be directly sensed and utilized by the entangled quantum entities; (2) quantum entanglement can persist in biological, chemical and physical systems at room and higher temperatures despite of quantum decoherence; and (3) quantum entanglement can influence chemical and biochemical reactions, other physical processes and micro- and macroscopic properties of all forms of matters. Therefore, it can be harnessed and developed into useful technologies to serve the mankind in many areas such as communication, engineering, health, medicine and recreation.

For example, using the apparatus and method developed in this invention I have discovered that the pH value of water in a detecting reservoir can be non-locally affected through manipulating water in a remote reservoir quantum-entangled with the water in the detecting reservoir.

For another example, using the apparatus and method developed in this invention I have further discovered that temperature of the water in said detecting reservoir can be non-locally affected through manipulating the water in said remote reservoir quantum-entangled with the water in the detecting reservoir and said temperature can change against that of local environment surrounding said detecting reservoir.

For yet another example, using the apparatus and method developed in this invention I have also discovered that gravity of the water in said detecting reservoir can be non-locally affected through manipulating the water in said remote reservoir quantum-entangled with the water in the detecting reservoir and the said gravity can change against local gravity surrounding said detecting reservoir.

Key to the present invention is an apparatus for generating a non-local effect which includes a target substance; an originating substance, said target substance being quantum-entangled with said originating substance; a mean for manipulating said originating substance, such that when said manipulation mean operates, said non-local effect is generated in said target substance through non-local processes mediated by quantum entanglement.

In one broad embodiment, the invention provides an apparatus that changes the physical property of a target substance quantum-entangled with an originating substance through non-local processes mediated by quantum entanglement.

In another broad embodiment, the invention provides a method for changing the physical property of one target substance quantum-entangled with an originating substance through non-local processes mediated by quantum entanglement.

In yet another broad embodiment, the invention provides an apparatus that changes the chemical property of a target substance quantum-entangled with an originating substance through non-local processes mediated by quantum entanglement.

In another broad embodiment, the invention provides a method for changing the chemical property of one target substance quantum-entangled with an originating substance through non-local processes mediated by quantum entanglement.

In yet another broad embodiment, the invention provides an apparatus that changes the biological property of a target biological system in vitro quantum-entangled with an originating substance through non-local processes mediated by quantum entanglement.

In yet another broad embodiment, the invention provides a method for changing the biological property of a target biological system in vitro quantum-entangled with an originating substance through non-local processes mediated by quantum entanglement.

In yet another broad embodiment, the invention provides an apparatus that changes the biological property of a target biological system in vivo quantum-entangled with an originating substance through non-local processes mediated by quantum entanglement.

In yet another broad embodiment, the invention provides a method for changing the biological property of a target biological system in vivo quantum-entangled with an originating substance through non-local processes mediated by quantum entanglement.

In yet another broad embodiment, the invention provides an apparatus that enables communications between two or more quantum-entangled systems separated by arbitrary distances through non-local processes mediated by quantum entanglement.

In another broad embodiment, the invention provides method for communicating between two or more quantum-entangled systems separated by arbitrary distances through non-local processes mediated by quantum entanglement.

One benefit of the present invention is that the physical and/or chemical properties such as pH values, temperatures and gravities of two or more quantum-entangled systems separated by arbitrary distances can be, in one broad embodiment, manipulated or modified for a desired purpose. A second benefit of the present invention is that the beneficial effects of chemical substances such as medications can be, in one broad embodiment, delivered to desired biological systems such as human patients from a remote location of arbitrary distance. A third benefit of the present invention is that two or more quantum-entangled systems separated by arbitrary distances can, in one broad embodiment, communicate among themselves without the assistance of any classical channels.

My invention may be more completely understood by reference to the following detailed description considered in connection with the accompanying drawings. However, it should be understood that the drawings are designed for purposes of illustration only and not as a definition of the limits of the invention.

### BRIEF DESCRIPTION OF THE DRAWINGS

FIG. 1 is a schematic view of an apparatus according to one embodiment producing physical and/or chemical non-local effect in the target substance, through manipulation of the originating substance quantum-entangled with said target substance, optionally measurable by an internal probe such as pH meter or thermometer for the purpose of monitoring and/or non-local communication.

FIG. 2 is a schematic view of several embodiments of the manipulating mean for manipulating the originating substance quantum-entangled with the target substance to produce non-local effect in said target substance through non-local processes mediated by quantum entanglement.

FIG. 3 is a schematic view of the apparatus illustrated in FIG. 1 producing physical and/or chemical non-local effect on said target substance, through manipulation of the originating substance quantum-entangled with said target substance, optionally measurable by an external probe such as analytical balance or spectrophotometer for the purpose of monitoring and/or non-local communication.

FIG. 4 is a schematic view of one method according to one embodiment for non-locally influencing a chemical reaction in a chemical system by first contacting said target substance with a chemical substance or mixture of chemical substances in said chemical system and then manipulating the originating substance quantum-entangled with said target substance.

FIG. 5 is a schematic view of another method according to one embodiment for non-locally influencing a biological system in vitro such as a cell culture by first contacting said target substance with said system in vitro and then manipulating the originating substance quantum-entangled with said target substance.

FIG. 6 is a schematic view of another method according to one embodiment for non-locally influencing a biological system in vivo such as a human body by first applying said target substance into said system in vivo and then manipulating the originating substance quantum-entangled with said target substance.

FIG. 7 is a schematic view of an apparatus according to one embodiment producing physical and/or chemical non-local effects in at least two targets, through manipulation of the originating substance quantum-entangled with target substances in said targets, respectively and optionally measurable by aforesaid internal probe such as the pH meter or the thermometers for the purpose of monitoring and/or non-local communication.

FIG. 8 is a schematic view of the apparatus illustrated in FIG. 7 producing physical and/or chemical non-local effects in the target substances, through manipulation of the originating substance quantum-entangled with said target substances, respectively and optionally measurable through aforesaid external probe for the purpose of monitoring and/or non-local communication.

FIG. 9 is a schematic view of the apparatus illustrated in FIG. 7 producing physical and/or chemical non-local effects in the target substances, through manipulation of the originating substance quantum-entangled with said target substances, respectively and optionally measurable through aforesaid internal probe and/or external probe for the purpose of monitoring and/or non-local communication.

FIG. 10 is a schematic view of one parallel combination of the apparatus illustrated in FIG. 1 for simultaneously transmitting two bits of information through said non-local effect from an originating location to a target location of arbitrary distance from said originating location.

FIG. 11 is a schematic view of one parallel combination of the apparatus illustrated in FIG. 3 for simultaneously transmitting two bits of information through said non-local effect from said originating location to said target location of arbitrary distance from said originating location.

FIG. 12 is a schematic view of yet another parallel combination of apparatuses illustrated in FIG. 1 and FIG. 3 for simultaneously transmitting two bits of information through said non-local effects from said originating location to the target location of arbitrary distance from said originating location.

FIG. 13 is a schematic view of one reverse combination of apparatus illustrated in FIG. 1 for exchanging two bits of information through said non-local effect between two locations separated by an arbitrary distance.

FIG. 14 is a schematic view of another reverse combination of apparatus illustrated in FIG. 3 for exchanging two bits of information through said non-local effect between said two locations separated by an arbitrary distance.

FIG. 15 is a schematic view of yet another reverse combination of apparatuses illustrated in FIG. 1 and FIG. 3 for exchanging two bits of information through said non-local effects between said two locations separated by an arbitrary distance.

FIG. 16 is a schematic view of an apparatus according to one embodiment producing non-local physical and/or chemical effects in a first substance, through quantum entanglement between said first substance and a second substance generated by a quantum entanglement generating source such as a laser or magnetic coil, optionally measurable by an internal and/or external probe such as pH meter.

FIG. 17 is a schematic view of a variation of the apparatus in FIG. 16 producing non-local physical and/or chemical effects in said first substance in which the said first substance and second substance are disposed in a reverse order of that shown in FIG. 16.

FIG. 18-19 and FIG. 22 are statistical chart views of experimental data obtained on target substance showing time series of changes in pH value, temperature or weight according to embodiment illustrated in FIG. 1 and FIG. 3 for detecting and measuring a physical and/or chemical non-local effect.

FIG. 20 and FIG. 21 are chart views of experimental data obtained on target substance showing time series of changes in temperature or weight according to embodiment illustrated in FIG. 1 and FIG. 3 for detecting and measuring a physical and/or chemical non-local effect.

FIGS. 23-25 are chart views of experimental data obtained on target substances showing time series of changes in temperature and weight respectively according to embodiment illustrated in FIG. 7-FIG. 9 for detecting and measuring physical and/or chemical non-local effects.

FIG. 26 is a chart view of experimental data obtained on target substance showing time series of changes in temperature according to embodiment illustrated in FIG. 4 and FIG. 5 for detecting and measuring a physical and/or chemical non-local effect.

FIG. 27 is a statistical chart view of experimental data obtained on target substance showing time series of changes in pH value according to embodiment illustrated in FIG. 16 for detecting and measuring a physical and/or chemical non-local effect.

### DETAILED DESCRIPTION OF THE INVENTION

The apparatus of the present invention in one broad embodiment includes the target substance, a first container holding said target substance; the originating substance, a second container holding said originating substance, said target substance being quantum-entangled with said originating substance; and said mean for manipulating said originating substance.

The said mean will be, depending on a particular use, any mean, such as a mean for cooling, heating, irradiating or adding a specific substance to said originating substance, which is capable of generating non-local effects in said target substance when said mean operates. The selection and operating specifications of the mean will vary according to the use. The person skilled in the art will be able readily to determine the appropriate mean and operating specifications of said mean, with only routine experimentation, for optimum performance of the specific use intended.

The said target or originating substance will be, depending on the use, a single substance or a mixture of several substances and has the physical forms of a liquid, gel, powder, solid or gas, or a mixture of these said forms. Again, the selection of the substance or specific mixture of substances and their precise concentrations will vary according to the use. It will, however, from the information herein, be well within the ability of a person of ordinary skill in the art to select the appropriate mixture of substances for the particular use intended by such person, with no more than routine experimentation.

The said first or second container will be any material and form capable of supportive functions such as a simple plastic frame, a glass or plastic bottle, or polymer matrix. The container will be optional if the substance or the mixture of substances will be made into an appropriate solid.

The said target substance and originating substance will be quantum-entangled through one of several quantum-entanglement processes discovered by me (Hu, H. P., & Wu, M. X. Photon induced non-local effect of general anesthetics on the brain. NeuroQuantology 4,17-31 (2006); Hu, H. P., & Wu, M. X. Non-local effects of chemical substances on the brain produced through quantum entanglement. Progress in Physics v3, 20-26 (2006)). For example, in one process a certain volume of a liquid, gel, gas, solid or a composition thereof such as water or a chemical solution in a suitable container will be exposed to electromagnetic radiation of a desired wavelength for a desired length of time before use. In a second process, the said liquid, gel, gas, solid or certain composition thereof such as water or a chemical solution in a suitable container will be simply left alone at a desired temperature for a certain period of time before use. Yet again, the selection and operating specifications of the quantum-entanglement processes will vary according to the use. The person skilled in the art will be able readily to determine the appropriate process and operating specifications of said process, with only routine experimentation, for optimum performance of the specific use intended.

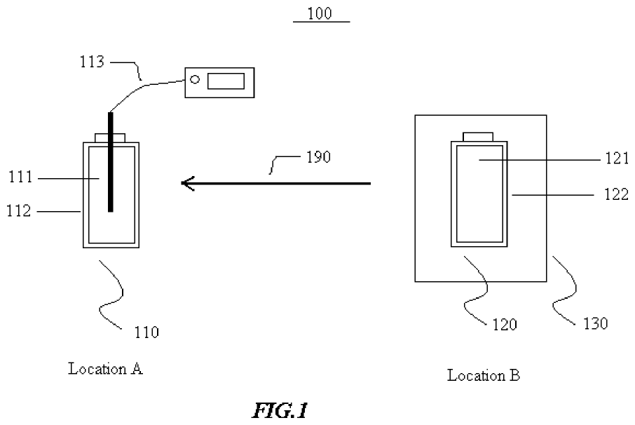

Considering first FIG. 1, the apparatus 100 of the present invention in one embodiment includes a target 110, a source 120 and said mean 130 for manipulating said source 120. Said target 110 further includes the target substance 111, said first container 112 holding said substance 111 and said internal probe 113 inserted into said container 112. Said source further including said originating substance 121 and said second container 122 holding said substance 121.

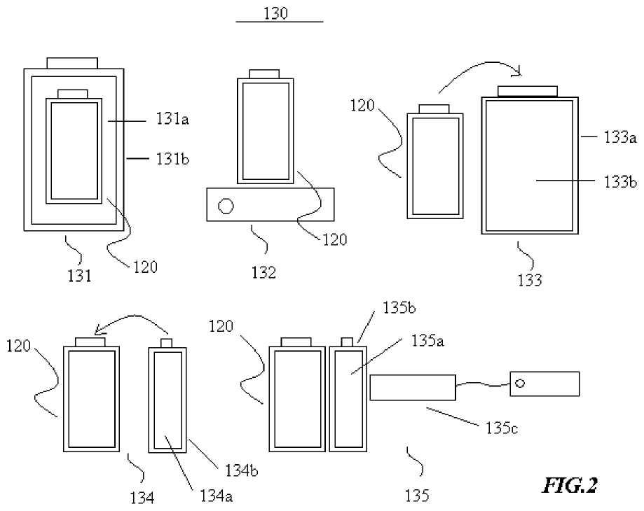

In one particular embodiment, the target substance 111 and originating substance 121 are quantum-entangled water prepared according to one of the said quantum-entanglement process, the internal probe is a traceable-calibration digital thermometer with a resolution of 0.001° C. and repeatability of 0.002° C. in liquid near 25° C., container 112 is a small flat glassware of the dimensions about 1″×4″×6″ (thickness, width, height) with a useful internal volume of about 250 ml, container 122 is a round plastic ware of the dimensions 2″×7″ (diameter, height) with a useful internal volume of about 350 ml, and the manipulation mean 130 is a particular embodiment of mean 131 shown in FIG. 2 which includes a 25-liter Dewar and 10-25 liters of liquid nitrogen filling said Dewar. The container 112 has a removable cap so that it can be filled, emptied, closed and fitted with the said probe 113. The container 122 also has a removable cap so that container 122 can be filled, emptied and closed. It will be understood, however, that the invention is not limited only to quantum-entangled water but also applies to other quantum-entangled media. It will be further understood that the internal probe is not limited only to the said digital thermometer but also applies to other internal probes such as pH meter and conductivity meter depending on a particular purpose.

To use the apparatus having this particular embodiment for a desired purpose such as non-local signaling, control of a device or manipulation of the physical and or chemical properties of the target substance, one disposes the said target 110 to a desired location A with well-controlled environment and the said source 120 to another desired location B, operates the manipulation mean 131 by submerging the container 122 containing substance 121 into the 25-litre Dewar filled with 10-25 liters of liquid nitrogen for a desired length of time whereby the target substance 111 are remotely influenced by the operation of the said manipulation mean through non-local process 190 mediated by quantum entanglement between the target substance 111 and originating substance 121, and records readings of said probe 113 both before and during the operation of the said mean 131 for a desired period of time depending on a desired purpose.

FIG. 2 shows several embodiments of the manipulation mean 130 including said mean 131 mentioned immediately above. Said mean 131 includes a substance 131 a and a container 131 b filled said substance 131 a such that when said source 120 is submerged into said substance 131 a a non-local effect is produced in the target 110 for an intended purpose through said non-local process mediated by quantum entanglement between the target substance 111 and originating substance 121. In the particular embodiment described immediately above, said container 131 b is a 25-liter Dewar and said substance 131 a is 10-25 liters of liquid nitrogen filling said Dewar.

Mean 132 shown in FIG. 2 includes a manipulation member 132 disposed beneath, above or next to or otherwise enclosing the source 120 such that when said member operates a non-local effect is produced in the target 110 for an intended purpose through said non-local process mediated by quantum entanglement between the target substance 111 and originating substance 121. In one particular embodiment, the said manipulation member 132 is a household gas or electric stove. In another particular embodiment, the said manipulation member 132 is a household microwave oven.

Mean 133 shown in FIG. 2 includes a substance 133a and a container 133b filled with said substance 133a such that when the originating substance 121 contacts said substance 133a a non-local effect is produced in the target 110 for an intended purpose through said non-local process mediated by quantum entanglement between the target substance 111 and originating substance 121. In one particular embodiment, said substance 133a is a desired acidic or alkaline solution such as concentrated HCl or NaOH solution and said container 133b is a glass container of an appropriate shape and size with a removable cap. In another particular embodiment, said substance 133a is a quantum-entangled large liquid mass such as 200 gallons of quantum entangled water and said container 133b is a large tank capable of holding said large liquid mass.

Mean 134 includes a substance 134a and a container 134b filled with said substance 134a such that when said substance 134a contacts the originating substance 121 a non-local effect is produced in the target 110 for an intended purpose through said non-local process mediated by quantum entanglement between the target substance 111 and originating substance 121. In one particular embodiment, said substance 134a is a desired acidic or alkaline solution such as concentrated HCl or NaOH solution and said container 134b is a glass container of a desired size with a removable cap.

Mean 135 includes a substance 135a, a container 135b filled with said substance 135a and disposed next to said contained 122, and an radiation member 135c disposed adjacent to said container 135b such that when said radiation member 135c operates said substance 135a gets quantum entangled with said substance 121 which, in turn, produces a non-local effect in the target 110 for an intended purpose through said non-local process mediated by quantum entanglement between the target substance 111 and originating substance 121. In one particular embodiment, said radiation member 135c is a laser of a desired wavelength and output. In another particular embodiment, said radiation member is a magnetic coil of desired output and an appropriate driving mechanism.

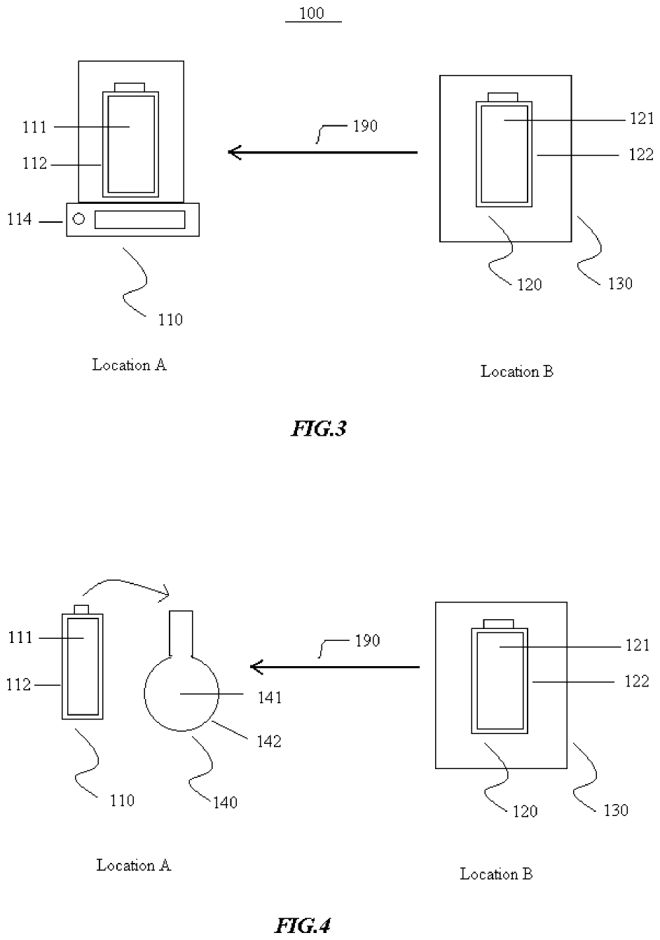

Considering next FIG. 3, the apparatus 100 of the present invention in one embodiment is a variation of the apparatus shown in FIG. 1 in which the internal probe 113 is replaced by an external probe 114 disposed beneath, above or next to said container 112. In one particular embodiment, the external probe 114 is an analytic balance with a capacity 210g, resolution 0.1 mg, repeatability 0.1 mg and sensitivity drift 3 PPM/° C., and other elements of the apparatus are the same as the ones described in the particular embodiment of the apparatus shown in FIG. 1. The said analytic balance may further include a windshield enclosing the weight pan. To use the apparatus having this particular embodiment, one carries out same steps as those for operating the particular embodiment of the apparatus shown in FIG. 1 with the exception that instead of recording readings of the internal probe 113, the digital thermometer, the readings of the external probe 114, the analytic balance, are recorded both before and during the operation of the said mean 130 for a desired period of time.

FIG. 4 illustrates a method according to one embodiment for non-locally influencing a chemical reaction in a chemical system 140 for a beneficial purpose. The essential steps include providing said quantum entangled target substance 111 and originating substance 121, a chemical substance or a mixture of chemical substances 141 and said manipulation mean 130; contacting said target substance 111 with said chemical substance or the mixture of chemical substances 141; and operating the manipulation mean 130; whereby the chemical reaction in the chemical system 140 is remotely influenced through said non-local process 190 mediated by quantum entanglement between the target substance 111 and originating substance 121.

FIG. 5 illustrates another method according to one embodiment for non-locally influencing a biological system in vitro 150 for a beneficial purpose. The essential steps include providing said quantum entangled target substance 111 and originating substance 121, a desired biological target in vitro 151 and a manipulation mean 130; applying said target substance 111 to said biological target in vitro 151; and operating the manipulation mean 130; whereby the said biological target in vitro 151 is remotely influenced through said non-local process 190 mediated by quantum entanglement between the target substance 111 and originating substance 121.

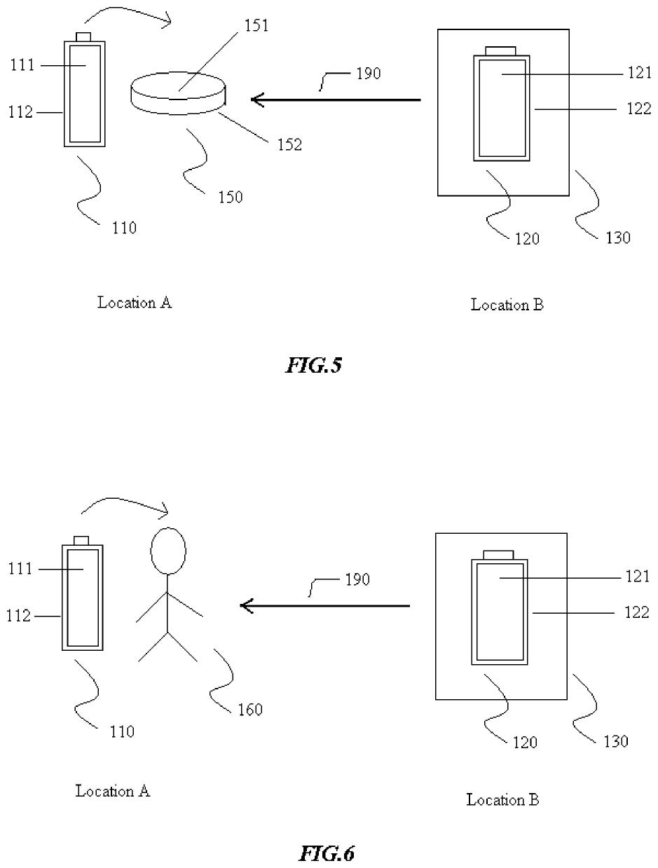

FIG. 6 illustrates yet another method according to one embodiment for non-locally influencing a biological object in vivo 160 for a beneficial purpose. The essential steps include providing said quantum entangled target substance 111 and originating substance 121, a desired biological object in vivo 160 and a manipulation mean 130; applying said target substance 111 to said biological object in vivo 160; and operating the manipulation mean 130; whereby the said biological object in vivo 160 is remotely influenced through said non-local process 190 mediated by quantum entanglement between the target substance 111 and originating substance 121.

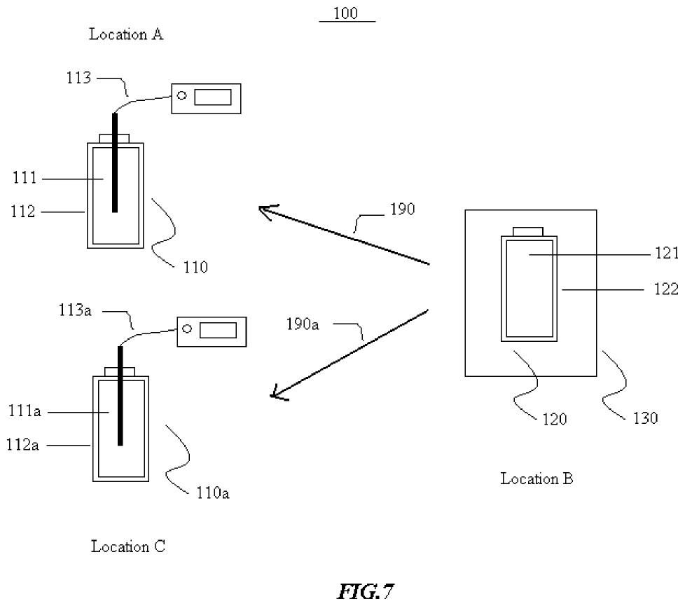

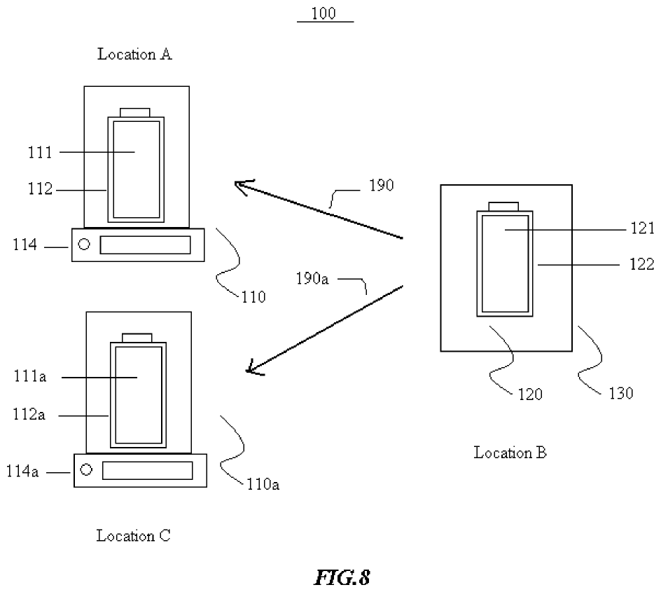

Considering next FIG. 7, the apparatus 100 of the present invention in one embodiment includes at least two targets 110 and 110a. This apparatus produces physical and/or chemical non-local effects in at least two target substances 111 and 111a, through manipulation of the originating substance 121 quantum-entangled with said target substances 111 and 111a, respectively measurable by aforesaid internal probe such as the pH meter or the thermometer for the purpose of monitoring and/or non-local communication.

To use the apparatus having said embodiment immediately above for a desired purpose such as non-local signaling to two locations or manipulation of the physical and/or chemical properties of said target substances, one disposes the said target 110 to a desired location A, the said target 110a to a desired location C and the said source 120 to another desired location B, operates the manipulation mean 130 for a desired length of time whereby the target substances 111 and 111a are remotely influenced by the operation of the said manipulation mean through non-local processes 190 and 190a respectively mediated by quantum entanglement of the target substances 111 and 111a with originating substance 121, and records readings of internal probes 113 and 113a both before and during the operation of the said mean 130 for a desired period of time depending on said desired purpose.

Considering next FIG. 8, the apparatus 100 of the present invention in one embodiment is a variation of the apparatus shown in FIG. 7 in which the internal probes 113 and 113a are replaced by external probes 114 and 114a respectively disposed beneath, above or next to containers 112 and 112a. To use the apparatus having this embodiment for a desired purpose, one carries out same steps as those for using the apparatus shown in FIG. 7 except that instead recording the readings of said internal probes 113 and 113a the readings of said external probes 114 and 114a are recorded.

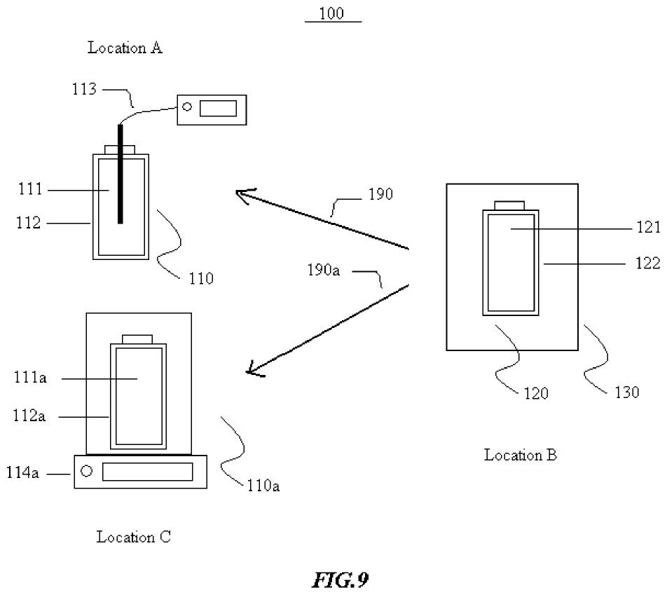

Considering next FIG. 9, the apparatus 100 of the present invention in one embodiment is yet another variation of the apparatus shown in FIG. 7 in which the internal probes 113a is replaced by external probes 114a disposed beneath, above or next to container 112a. To use the apparatus having this embodiment for a desired purpose, one carries out same steps as those for using the apparatus shown in FIG. 7 except that instead recording the readings of said internal probe 113a the readings of said external probe 114a are recorded.

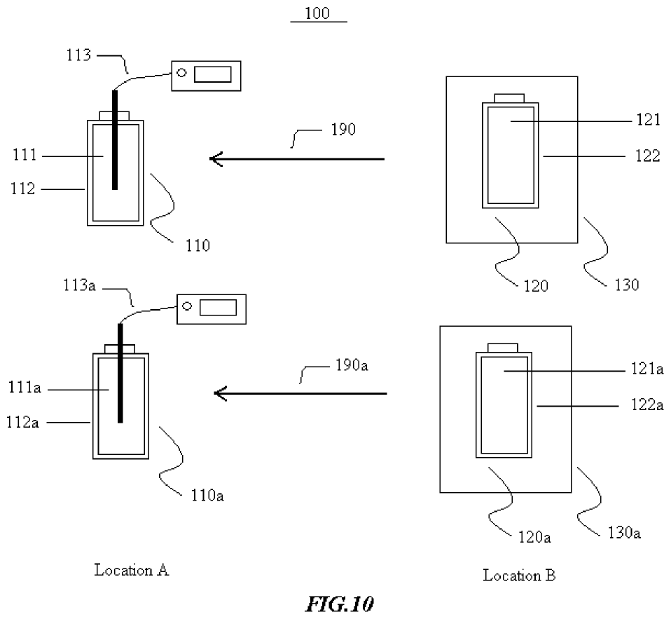

Considering next FIG. 10, the apparatus 100 of the present invention in one embodiment is a parallel combination of two apparatuses shown in FIG. 1 with each respectively including a target 110 or 110a and a source 120 or 120a. This apparatus can simultaneously produces physical and/or chemical non-local effects in said respective target 111 or 111a, through respective manipulation of the originating substance 121 or 121 a quantum-entangled with said target substances 111 or 111a, measurable by aforesaid internal probe such as the pH meter or the thermometer for the purpose of monitoring and/or non-local communication.

To use the apparatus having said embodiment immediately above for a desired purpose such as simultaneous non-local transmission of two bits of information from one location to another, one disposes the said targets 110 and 110a to a desired location A and the said sources 120 and 120a to another desired location B, operates the manipulation means 130 and 130a for a desired length of time whereby the target substances 111 and 111a are remotely influenced by the operation of the said manipulation means 130 and 130a through non-local processes 190 and 190a respectively mediated by quantum entanglement between said target substances 111 and originating substance 121 and that between said target substances 111a and originating substance 121a, and records readings of internal probes 113 and 113a both before and during the operations of the said mean 130 and 130a for a desired period of time depending on said desired purpose.

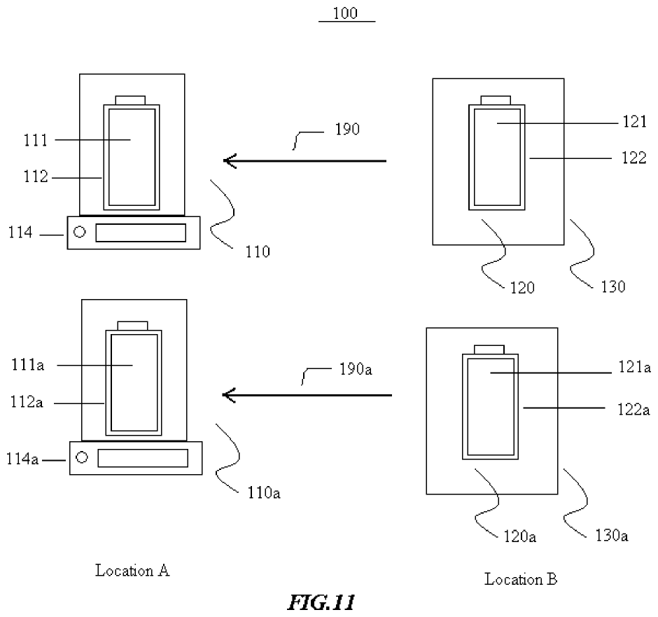

Considering next FIG. 11, the apparatus 100 of the present invention in one embodiment is a variation of the apparatus shown in FIG. 10 in which the internal probes 113 and 113a are replaced by external probes 114 and 114a respectively disposed beneath, above or next to containers 112 and 112a. To use the apparatus having this embodiment for a desired purpose, one carries out same steps as those for using the apparatus shown in FIG. 10 except that instead recording the readings of said internal probes 113 and 113a the readings of said external probes 114 and 114a are recorded.

Considering next FIG. 12, the apparatus 100 of the present invention in one embodiment is yet another variation of the apparatus shown in FIG. 10 in which the internal probes 113a is replaced by external probes 114a disposed beneath, above or next to container 112a. To use the apparatus having this embodiment for a desired purpose, one carries out same steps as those for using the apparatus shown in FIG. 10 except that instead recording the readings of said internal probe 113a the readings of said external probe 114a are recorded.

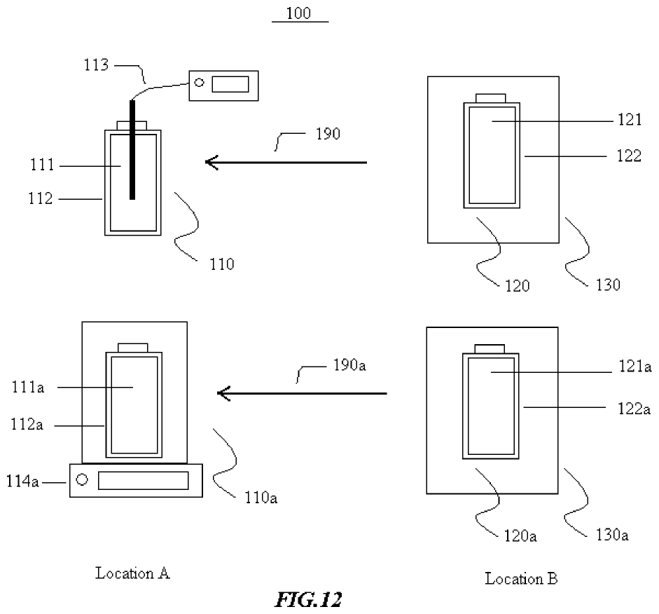

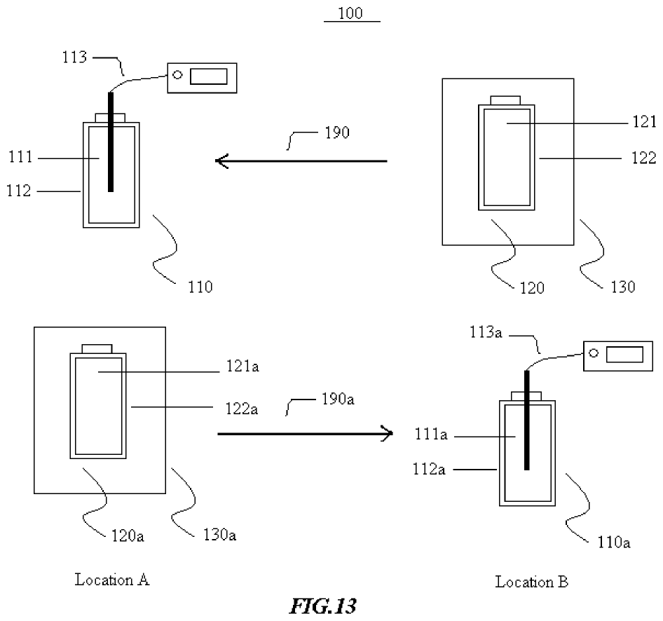

Considering next FIG. 13, the apparatus 100 of the present invention in one embodiment is a reverse combination of at least two apparatuses shown in FIG. 1 with each including a target 110 or 110a and a source 120 or 120a. This apparatus produces physical and/or chemical non-local effects in said respective target substance 111 or 111a, through manipulation of the originating substance 121 or 121 a quantum-entangled with said target substances 111 or 111a, measurable by aforesaid internal probe such as the pH meter or the thermometer for the purpose of monitoring and/or non-local communication.

To use the apparatus having said embodiment immediately above for a desired purpose such as non-local exchange of information between two locations, one disposes the said target 110 and source 120a to a desired location A and the said sources 120 and target 110a to another desired location B, operates the manipulation means 130 and 130a in a desired sequence and for a desired length of time whereby the target substances 111 and 111a are remotely influenced by the operation of the said manipulation means 130 and 130a through non-local processes 190 and 190a respectively mediated by quantum entanglement between said target substances 111 and originating substance 121 and that between said target substances 111a and originating substance 121a, and records readings of internal probes 113 and 113a both before and during the operations of the said mean 130 and 130a for a desired period of time depending on said desired purpose.

Considering next FIG. 14, the apparatus 100 of the present invention in one embodiment is a variation of the apparatus shown in FIG. 13 in which the internal probes 113 and 113a are replaced by external probes 114 and 114a respectively disposed beneath, above or next to containers 112 and 112a. To use the apparatus having this embodiment for a desired purpose, one carries out same steps as those for using the apparatus shown in FIG. 13 except that instead recording the readings of said internal probes 113 and 113a the readings of said external probes 114 and 114a are recorded.

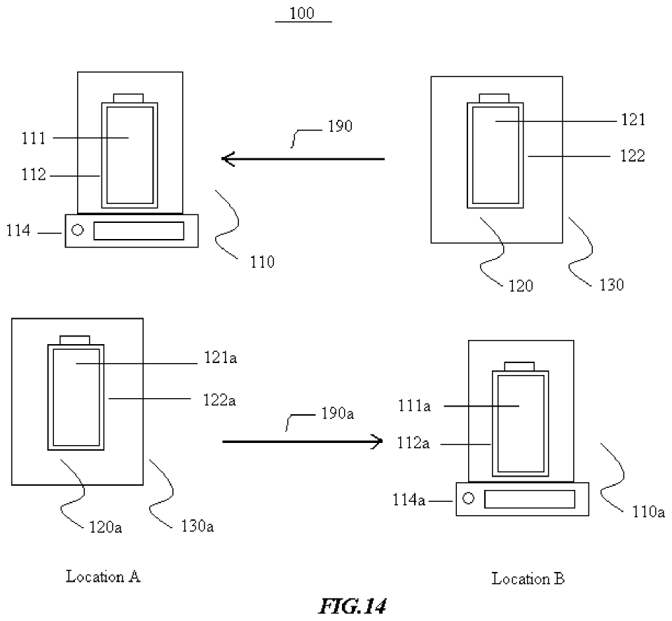

Considering next FIG. 15, the apparatus 100 of the present invention in one embodiment is yet another variation of the apparatus shown in FIG. 13 in which the internal probes 113a is replaced by external probes 114a disposed beneath, above or next to container 112a. To use the apparatus having this embodiment for a desired purpose, one carries out same steps as those for using the apparatus shown in FIG. 13 except that instead recording the readings of said internal probes 113 and 113a the readings of said external probes 114 and 114a are recorded.

Considering next FIG. 16, the apparatus 200 of the present invention in one embodiment includes a first substance 211, a second substance 221 and a quantum entanglement generating source 230 for quantum-entangling said first and second substances 211 and 221. Said apparatus further includes a first container 212 for holding said first substance 211, a second container 222 for holding said second substance 221 and an optional internal probe 213 inserted into said container 212.

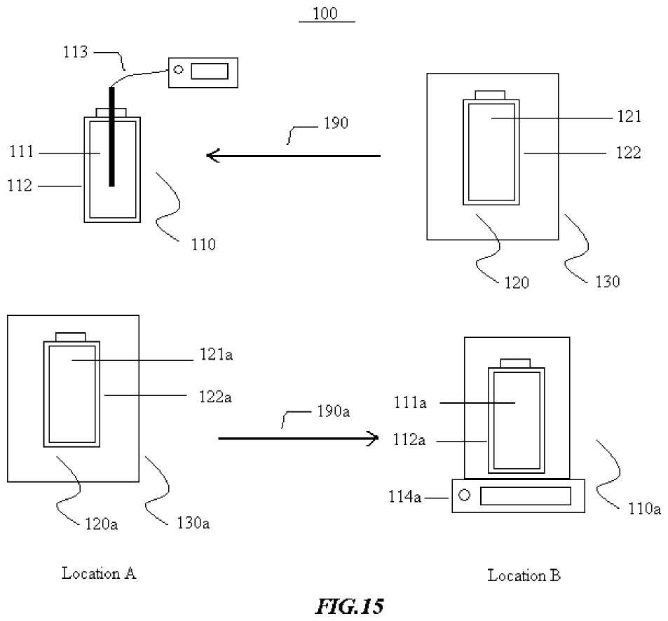

To use the apparatus having said embodiment immediately above for a desired purpose such as non-local manipulation of the physical and/or chemical properties of said first substance 211, one disposes the said first container 212 filled with said first substance 211 between said second container 222 filled with said second substance 221 and said generating source 230, operates said generating source 230 for a desired length of time whereby said first and second substances 211 and 221 are quantum entangled and said physical and/or chemical properties of said first substance 211 are affected by said second substance 221 through non-local processes mediated by said quantum entanglement, and optionally records readings of said internal probe 213 both before and during the operation of the said generating source 230 for a desired period of time depending on said desired purpose. In one particular embodiment, said generating source 230 is a laser of a desired wavelength and output. In another particular embodiment, said generating source 230 is a magnetic coil of desired output and an appropriate driving mechanism.
Considering next FIG. 17, the apparatus 200 of the present invention in one embodiment is a variation of the apparatus shown in FIG. 16 in which the said first substance 211 and second substance 221 are disposed in a reverse order of that in FIG. 16. To use the apparatus having this embodiment for a desired purpose, one carries out same steps as those for using the apparatus shown in FIG. 16 except that said second container 212 filled with said second substance 221 is disposed between said first container 212 filled with said first substance 211 and said generating source 230.

It will be appreciated that the particular features of the methods and apparatuses illustrated and described herein may be employed separately or in combination in any suitable manner so as to enhance the beneficial purposes. Those skilled in the art will also of course recognize that substitutions can be made, as long as the changes do not materially affect the ability of the methods and apparatuses disclosed herein.

Various experimental studies with the apparatus and method disclosed herein were carried out to evaluate the non-local effects produced on the target substance, in particular, water, using physical and/or chemical observables such as pH, temperature and gravity measured with high-precision instruments. My motivation for measuring pH change of water in one reservoir, while manipulating water in a remote reservoir quantum-entangled with the former, is to investigate whether and how pH value in the water being measured shifts under non-local influences. My motivation for measuring temperature variation of water in one reservoir, while manipulating water in a remote reservoir quantum-entangled with the former, is to investigate whether and how the thermodynamics of water being measured changes under non-local influences. My motivation for measuring gravity change of one reservoir of water, while manipulating water in a remote reservoir quantum-entangled with the former, is to investigate whether gravity is a non-local effect associated with quantum entanglement.

Further, the said experiments were achieved with the aids of high-precision analytical instruments. They include an Ohaus Voyager analytical balance with capacity 210g, resolution 0.1 mg, repeatability 0.1 mg and sensitivity drift 3 PPM/° C., a Control Company traceable-calibration digital thermometer with resolution 0.001° C. and repeatability 0.002° C. near 25° C. in liquid such as water (estimated from calibration data provided), and a Hanna microprocessor pH meter Model 213 with resolution 0.001 and repeatability 0.002. The other key device is a 25-litre Dewar filled with liquid nitrogen and positioned remotely at a desired distance which not only provided the drastic changes in the water being manipulated but also served as a natural Faraday cage blocking any possible electromagnetic influence between the water being measured and the water being manipulated. Also important to the experiments described herein was the stable environment found in an underground room which shields many external noises such as mechanical vibration, air turbulence and large temperature change.

Quantum-entangled stock water in individual volumes of 500 m/or similar quantities was prepared as described previously which might then be split into smaller volumes or combined into larger ones based on needs(Hu, H. P., & Wu, M. X. Photon induced non-local effect of general anesthetics on the brain. NeuroQuantology 4, 17-31 (2006); Hu, H. P., & Wu, M. X. Non-local effects of chemical substances on the brain produced through quantum entanglement. Progress in Physics v3, 20-26 (2006)). Briefly, in one procedure 500 m/fresh tap water in a closed plastic reservoir was exposed to microwave radiation in a 1500 W microwave oven for 2 min and then left in room temperature for 24 hours before use. In a second procedure 500 m/fresh tap water in the closed plastic reservoir was exposed to audio-frequency radiations of a 20 W magnetic coil for 30 min and then left in room temperature for 24 hours before use. In a third procedure, 500 m/bottled natural water was simply left in room temperature for at least 30 days before use. In a fourth procedure, 500 m/bottled distilled water was simply left in room temperature for at least 30 days before use. It was found previously that the stock water prepared according to these procedures is quantum-entangled (Hu, H. P., & Wu, M. X. Photon induced non-local effect of general anesthetics on the brain. NeuroQuantology 4, 17-31 (2006); Hu, H. P., & Wu, M. X. Non-local effects of chemical substances on the brain produced through quantum entanglement. Progress in Physics v3, 20-26 (2006)).

In the first key experimental setup, the apparatus illustrated in FIG. 9 having following particular embodiment was used: (1) the external probe 114a is the Ohaus Voyager analytical balance calibrated internally and stabilized in the underground room for more than one week before use; (2) the container 112a is a tightly closed round plastic reservoir of the dimensions 2″×7″ (diameter, height) with a useful internal volume of about 350 m/and a removal cap; (3) the target substance 111a is 175 m/water split from 500 m/quantum-entangled stock water and is placed on the wind-shielded pan of the balance with 1-inch white foam in between as insulation; (4) the container 112 is tightly closed round glass reservoir the dimensions 2″×4″ (diameter, height) with a useful internal volume of about 125 m/and a removal cap; (5) the target substance 111 is 75 m/water split from said 500 m/stock water; (6) the internal probe 113 is the Control Company traceable-calibration digital thermometer and/or the Hanna microprocessor pH meter Model 213 placed into the middle of the container 112; (6) the container 122 another tightly closed round plastic reservoir of the dimensions 2″×7″ (diameter, height) with a useful internal volume of about 350 m/and a removal cap; (7) the originating substance 121 is 250 m/water split from said 500 m/stock water; and (8) the manipulation mean 130 includes a 25-liter Dewar disposed at a distance of 50 or 500 feet from the targets 110 and 110a, and 15-25 liters of liquid nitrogen filling said Dewar.

Experiments with the above first setup carried out in the following steps: (1) prepare the 500 m/quantum-entangled stock water, divide the same into 175 m/, 75 m/and 250 m/portions and put them into their respective containers 112a, 112, and 122 described above; (2) set up the experiment according to FIG. 9 and let the instruments to stabilize for 30 min before any measurements is taken; (3) record for 20 min minute-by-minute changes of pH value and/or temperature of the water in the container 112 and weight of the container 112a with water before submerging the container 122 into said liquid nitrogen; (4) submerge the container 122 containing water 121 into said liquid nitrogen for 15 min or another desired length of time and record the readings of the internal and external probes 113 and 114a as before; and (5) take the container 122 out of the said liquid nitrogen, thaw the same in warm water for 30 min or longer and, at the same time, record the readings of the internal and external probes 113 and 114a as before. Control experiments were carried out in same steps with nothing done to the water 121 in the container 122.

Different variations of the above first setup were also used in the experiments. In one variation, the closed plastic container 122 was replaced with a metal container and instead of freeze-thaw treatment the water 121 in the metal container was quickly heated to boiling within 4-5 minutes and then cooled in cold water. In a second variation, the gravity portion of the experiment was eliminated and the water in the containers 112 and 112a were combined and put into a closed thermal flask, which prevents heat exchange between the water being measured and its local environment, for pH and temperature measurements. In a third variation, the gravity portion of the experiment was eliminated and the water in the containers 112 and 112a were combined and put into another plastic container for temperature, while said water 121 was, first, added with 5 m/concentrated HCl (38% by weight), second, added with 20 g NaOH powder, and, third, transferred to a metal container and heated to boiling on a stove. In a fourth variation, the 25-liter Dewar containing liquid nitrogen was replaced by a large water tank located 20-feet above the underground room which contained 200-gallon tap water sitting in room temperature for months and, instead of submersion, the water 121 in the container 122 was poured into the large water tank the purpose of which was to quantum-entangle the poured water with the water in the large tank. In a fifth variation, the gravity portion of the experiment was eliminated and the water in the containers 112 and 112a were combined and put into said plastic container which was moved to a location more than 50 miles away from the Dewar for temperature measurement.

In the second setup, the apparatus illustrated in FIG. 16 having following particular embodiment was used: (1) the quantum entanglement generating source 230 was a red laser with a 50 mWoutput and wavelengths 635 nm-675 nm; (2) the first container 212 was a flat glass reservoir of the dimensions 1″×4″×6″ (thickness, width, height) with a useful internal volume of about 250 ml disposed next to said laser 230; (3) the first substance 211 was 200 m/tap water filling said container 212 and sit in room temperature for more than a week without air exchange before use; (4) the internal probe 213 was the Hanna microprocessor pH meter Model 213 placed into the middle of the said flat glass container 212 which was closed to prevent air exchange; (5) the second container 222 was a tightly closed round glass reservoir of the dimensions 2″×4″ (diameter, height) with a useful internal volume of about 125 m/disposed at a distance of 500 cm from the container 212; and (6) the second substance 221 was 100 m/concentrated HCl (38% by weight).

Experiments with the above second setup were carried out as follows: (1) prepare the 200 m/tap water and set up the experiment according to FIG. 16; (2) turn on and operate said laser 230 such that the laser beam first passes through said first container 212 and then gets scattered on a nearby concrete wall; (3) let said pH meter 213 to stabilize for 30 min before any measurement is taken; (4) record for 10 min. minute-by-minute changes of pH value of water in said container 212; and (5) dispose said container 222 containing said second substance 221 on the path of the laser beam and at said distance of 500 cm from said container 212; and (6) record for 60 min or longer minute-by-minute readings from said pH meter 213. Control experiments were carried out in same steps without the presence of HCl.

Tables 1, 2 and 3 show sample data obtained from experiments conducted with the first setup on one batch of quantum-entangled water in which the manipulation mean 130 was at said distance of 50 or 500 feet away from the water 111 and 111a being measure and said water was simply individually bottled natural water with a shelf time of more than 90 days. Similar results were also obtained with water prepared according to other quantum entanglement methods mentioned above and other quantum-entangled liquid such as olive oil, alcohol, distilled water and Coca Cola as discussed later. The different distances of the manipulation mean from the target in the underground room where most measurements were done made no differences with respect to the non-local effects observed.

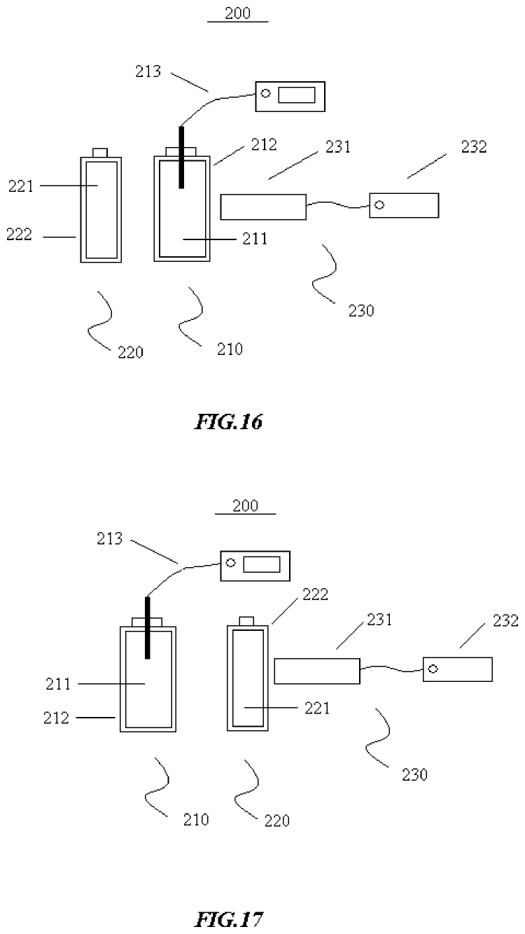

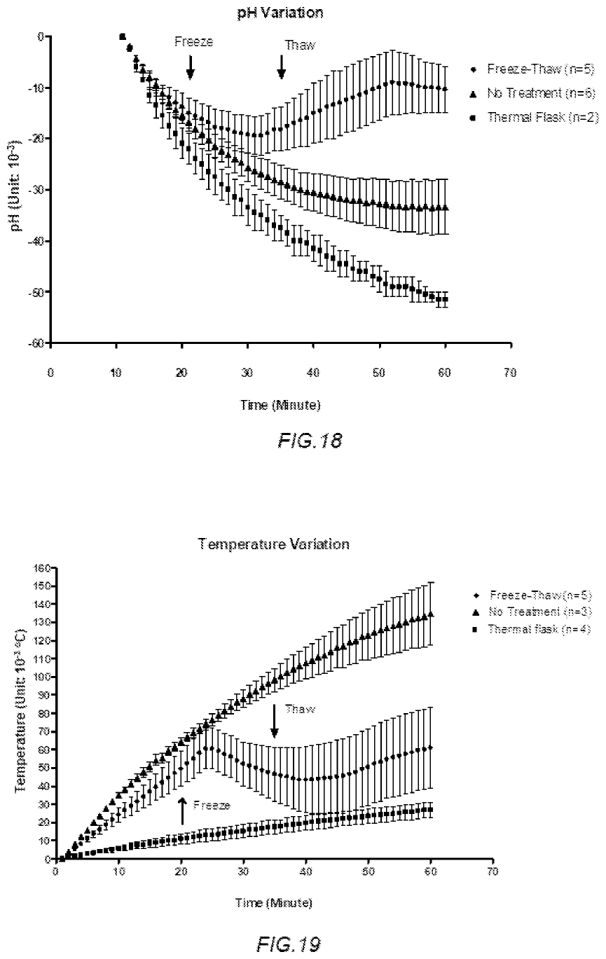

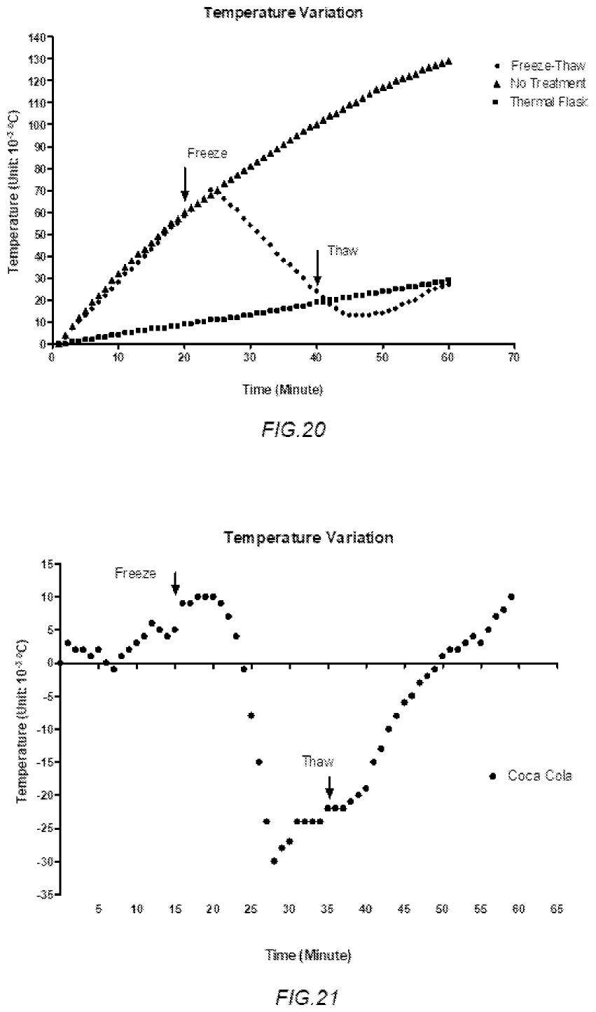

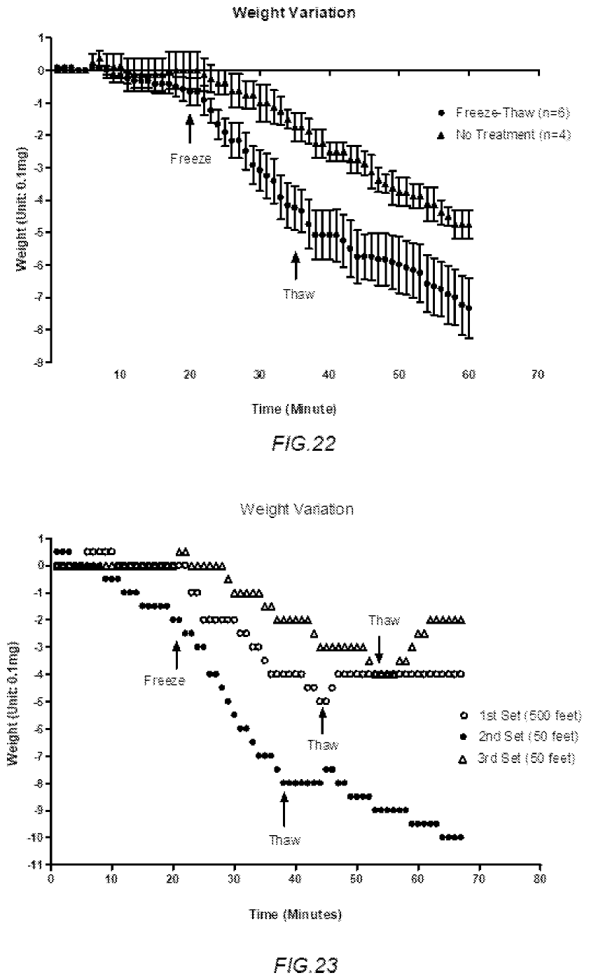

FIG. 18, FIG. 19 and FIG. 22 show statistical chart views of data sets obtained from experiments conducted with the first setup on one batch of quantum-entangled water in which the manipulation mean 130 was at said distance of 50 feet away from the water 111 and 111a being measure and said water was simply individually bottled natural water with a shelf time of more than 90 days. FIG. 20 shows chart view of one sample data set obtained on water. FIG. 21 shows chart view of one sample data set obtained on Coca Cola.

Table 1 shows one data set on the pH values of said target substance 111, the 75 m/water, in said container 112 during the three stages of manipulation of said originating substance 121, the 250 m/water, in said container 122 plus pH values obtained in the control experiment and with said thermal flask in said second variation of the first setup:

TABLE 1

| Time | Freeze-Thaw | | Control | Thermal Flask | |
|---|---|---|---|---|---|
| (Min) | pH (Unit: 10⁻³) | | pH (Unit: 10⁻³) | pH (Unit: 10⁻³) | |
| 11 | | | 0 | | 0 |
| 12 | | 0 | −3 | | −3 |
| 13 | | −4 | −6 | | −7 |
| 14 | | −7 | −9 | | −9 |
| 15 | | −9 | −11 | | −13 |
| 16 | | −12 | −13 | | −16 |
| 17 | | −13 | −14 | | −18 |
| 18 | | −16 | −16 | | −20 |
| 19 | | −17 | −17 | | −22 |
| 20 | Freeze | −19 | −19 | Freeze | −24 |
| 21 | | −23 | −21 | | −25 |
| 22 | | −24 | −22 | | −27 |
| 23 | | −25 | −22 | | −28 |
| 24 | | −26 | −24 | | −30 |
| 25 | | −28 | −25 | | −31 |
| 26 | | −29 | −26 | | −32 |
| 27 | | −29 | −28 | | −33 |
| 28 | | −30 | −28 | | −35 |
| 29 | | −31 | −29 | | −35 |
| 30 | | −31 | −30 | | −37 |
| 31 | | −32 | −31 | | −38 |
| 32 | | −32 | −32 | | −38 |
| 33 | | −32 | −32 | | −39 |
| 34 | | −32 | −33 | | −40 |
| 35 | Thaw | −32 | −33 | Thaw | −40 |
| 36 | | −32 | −33 | | −41 |
| 37 | | −32 | −34 | | −43 |
| 38 | | −32 | −34 | | −43 |
| 39 | | −32 | −34 | | −43 |
| 40 | | −31 | −34 | | −44 |
| 41 | | −30 | −34 | | −45 |
| 42 | | −29 | −34 | | −46 |
| 43 | | −29 | −35 | | −46 |
| 44 | | −27 | −35 | | −47 |
| 45 | | −26 | −35 | | −47 |
| 46 | | −25 | −35 | | −48 |
| 47 | | −24 | −35 | | −48 |
| 48 | | −23 | −36 | | −48 |
| 49 | | −22 | −36 | | −49 |
| 50 | | −21 | −37 | | −50 |
| 51 | | −21 | −37 | | −51 |
| 52 | | −21 | −38 | | −51 |
| 53 | | −21 | −38 | | −51 |
| 54 | | −21 | −38 | | −51 |
| 55 | | −21 | −38 | | −52 |
| 56 | | −21 | −38 | | −52 |
| 57 | | −21 | −38 | | −52 |
| 58 | | −21 | −38 | | −52 |
| 59 | | −21 | −38 | | −53 |
| 60 | | −21 | −38 | | −53 |

FIG. 18 shows statistical chart view of several pH data sets obtained on said target substance 111, the 75 m/water, in said container 112 during the three stages of manipulation of said originating substance 121, the 250 m/water, in said container 122 plus pH values obtained in the control experiment and with said thermal flask in said second variation of the first setup.

As shown in Table 1 and FIG. 18, within minutes after said container 122 was submerged into liquid nitrogen, during which the temperature of said water 121 being manipulated would drop from about 25° C. to −193° C., the pH value of said water 111 in said container 112 steadily stopped dropping and then started rising, but about 20 min after the frozen water 121 was taken out of liquid nitrogen and thawed in warm water the pH value of said water 111 steadily leveled off and started dropping again. In Table 11 and FIG. 18, the pH value at the starting point of measurement is set to zero.

In contrast, as shown in Table 1 and FIG. 18, the control experiments did not show such dynamics. It is known that the pH value of water increases as its temperature goes down to 0° C. Therefore, the pH value of said water 111 being measured goes in the same direction as said water 121 when the latter is manipulated. The difference in pH values from control in which no freeze-thaw was done at the point of thawing is about 0.010. However, in the second variation of the first setup, when said water 111 and 111a being measured were merged and kept in said thermal flask to prevent heat exchange with the local environment , no effect on pH value of said water 111 and 111a was observed under freeze-thaw treatment of said water 121. Statistical analysis performed on all data collected after freezing for 10 min show that the results are significantly different under these different treatments/settings as shown in FIG. 18.

Table 2 shows one data set on temperature variations of said target substance 111, the 75 m/water, in said container 112 during the three stages of manipulation of said originating substance 121, the 250 m/water, in said container 122 plus temperature variations obtained in the control experiment and with said thermal flask in said second variation of the first setup:

TABLE 2

| | Freeze-Thaw | | Control | Thermal Flask | |
|---|---|---|---|---|---|
| Time | Temp | | Temp | Temp | |
| (Minute) | (Unit: 10⁻³° C.) | | (Unit: 10⁻³° C.) | (Unit: 10⁻³° C.) | |
| 1 | | 0 | 0 | | 0 |
| 2 | | 3 | 4 | | 0 |
| 3 | | 7 | 8 | | 1 |
| 4 | | 10 | 12 | | 1 |
| 5 | | 13 | 15 | | 2 |
| 6 | | 16 | 19 | | 2 |
| 7 | | 19 | 22 | | 3 |
| 8 | | 22 | 25 | | 3 |
| 9 | | 25 | 29 | | 4 |
| 10 | | 28 | 32 | | 4 |
| 11 | | 32 | 35 | | 5 |
| 12 | | 34 | 38 | | 5 |
| 13 | | 37 | 41 | | 6 |
| 14 | | 40 | 43 | | 6 |
| 15 | | 43 | 46 | | 7 |
| 16 | | 46 | 49 | | 7 |
| 17 | | 50 | 52 | | 7 |
| 18 | | 53 | 55 | | 8 |
| 19 | | 55 | 57 | | 8 |
| 20 | Freeze | 58 | 60 | Freeze | 9 |
| 21 | | 61 | 62 | | 9 |
| 22 | | 63 | 64 | | 10 |
| 23 | | 66 | 66 | | 10 |
| 24 | | 70 | 68 | | 11 |
| 25 | | 69 | 70 | | 11 |
| 26 | | 66 | 73 | | 11 |
| 27 | | 63 | 75 | | 12 |
| 28 | | 61 | 77 | | 12 |
| 29 | | 57 | 79 | | 13 |
| 30 | | 54 | 81 | | 13 |
| 31 | | 51 | 83 | | 14 |
| 32 | | 48 | 85 | | 14 |
| 33 | | 45 | 87 | | 15 |
| 34 | | 41 | 89 | | 15 |
| 35 | | 38 | 91 | | 16 |
| 36 | | 36 | 93 | | 16 |
| 37 | | 33 | 95 | | 17 |
| 38 | | 30 | 97 | | 17 |
| 39 | | 26 | 99 | | 18 |
| 40 | Thaw | 24 | 100 | Thaw | 19 |
| 41 | | 21 | 102 | | 19 |
| 42 | | 18 | 104 | | 20 |
| 43 | | 16 | 105 | | 20 |
| 44 | | 14 | 107 | | 21 |
| 45 | | 13 | 109 | | 21 |
| 46 | | 13 | 110 | | 22 |
| 47 | | 13 | 112 | | 22 |
| 48 | | 13 | 114 | | 23 |
| 49 | | 14 | 116 | | 23 |
| 50 | | 14 | 117 | | 24 |
| 51 | | 15 | 118 | | 24 |
| 52 | | 16 | 120 | | 25 |
| 53 | | 18 | 121 | | 25 |
| 54 | | 19 | 122 | | 26 |
| 55 | | 20 | 123 | | 26 |
| 56 | | 22 | 125 | | 27 |
| 57 | | 24 | 126 | | 27 |
| 58 | | 25 | 127 | | 28 |
| 59 | | 26 | 128 | | 28 |
| 60 | | 27 | 129 | | 29 |

FIG. 19 shows statistical chart view of several temperature data sets obtained on said target substance 111, the 75 m/water, in said container 112 during the three stages of manipulation of said originating substance 121, the 250 m/water, in said container 122 plus temperature variations obtained in the control experiment and with said thermal flask in said second variation of the first setup.

As shown in Table 2, FIG. 19, and FIG. 20 which is chart view of the data set in Table 2, before the submersion of said container 122 into liquid nitrogen the temperature of said water 111 in said container 112 rose in small increments due to, by design, the slight temperature difference between the local environment and said water 111 in said container 112; but within about 4-5 minutes after said container 122 was submerged into liquid nitrogen, during which the temperature of said water 121 being manipulated would drop from about 25° C. to −193° C., the temperature of said water 111 in said container 112 first stopped rising and then steadily dropped in small increments; and then within about 4-5 minutes after said frozen water 121 was taken out of liquid nitrogen and thawed in warm water the temperature of said water 111 in said container 112 first stopped dropping and then steadily rose again in small increments. In Table 2, FIG. 19 and FIG. 20, the temperature at the starting point of measurement is set to zero.

In contrast, as shown in Table 2, FIG. 19 and FIG. 20, the control experiments did not show such dynamics. The temperature difference from control in which no freeze-thaw was done at the point of thawing is about 0.05° C. However, in the second variation of the first setup, when said water 111 and 111a being measured were merged and kept in said thermal flask to prevent heat exchange with the local environment, no dropping of temperature were observed under freeze-thaw treatment of said water 121. Statistical analysis performed on all data collected after freezing for 10 min show that the results are significantly different under these different treatments/settings as shown in FIG. 19.

The applicant also found that other liquids such as olive oil, alcohol, Coca Cola and distilled water also showed similar qualitative temperature variations under the said freeze-thaw treatment. Furthermore, experiments conducted with the fifth variation of the first setup in which the temperature measurement done at a location more than 50 miles way from the manipulation mean 130 also show results similar to those obtained at distances of 50 and 500 feet respectively.

FIG. 21 shows chart view of one temperature data set obtained on said target substance 111, 75 m/Coca Cola, in said container 112 during the three stages of manipulation of said originating substance 121, the 250 m/Coca Cola, in said container 122.

Columns 2, 3 and 4 in Table 3 respectively show one data set on: (1) weight variations of said target substance 111a, the 175 m/water, in said container 112a during the three stages of manipulation of said originating substance 121, the 250 m/water, in said container 122; (2) temperature variations of said target substance 111, the 75 m/water, in said container 112, simultaneous to the weight variations, during the same three stages of manipulation; and (3) weight variations obtained in the control experiment:

TABLE 3

| | Freeze-Thaw | | Freeze-Thaw | | Control |
|---|---|---|---|---|---|
| Time | Weight | | Temp | | Weight |
| (Min) | (Unit: 0.1 mg) | | (Unit: 10⁻³° C.) | | (Unit: 0.1 mg) |
| 1 | | 0.5 | | 0 | 0 |
| 2 | | 0.5 | | 2 | 0 |
| 3 | | 0.5 | | 3 | 0 |
| 4 | | 0 | | 5 | 0 |
| 5 | | 0 | | 6 | 0 |
| 6 | | 0 | | 7 | 0 |
| 7 | | 0 | | 8 | 0 |
| 8 | | 0 | | 9 | −0.5 |
| 9 | | −0.5 | | 10 | −0.5 |
| 10 | | −0.5 | | 11 | −0.5 |
| 11 | | −0.5 | | 12 | −1 |
| 12 | | −1 | | 12 | −1 |
| 13 | | −1 | | 13 | −1 |
| 14 | | −1 | | 13 | −1 |
| 15 | | −1.5 | | 14 | −1 |
| 16 | | −1.5 | | 15 | −1 |
| 17 | | −1.5 | | 15 | −1 |
| 18 | Freeze | −1.5 | Freeze | 16 | −1 |
| 19 | | −1.5 | | 16 | −1 |
| 20 | | −2 | | 17 | −1 |
| 21 | | −2 | | 17 | −1 |
| 22 | | −2.5 | | 18 | −1 |
| 23 | | −2.5 | | 20 | −1 |
| 24 | | −3 | | 22 | −1 |
| 25 | | −3 | | 22 | −1 |
| 26 | | −4 | | 21 | −1.5 |
| 27 | | −4 | | 19 | −1.5 |
| 28 | | −4.5 | | 17 | −1.5 |
| 29 | | −5 | | 14 | −1.5 |
| 30 | | −5.5 | | 11 | −2 |
| 31 | | −6 | | 9 | −2 |
| 32 | | −6 | | 6 | −2 |
| 33 | | −6.5 | | 3 | −2 |
| 34 | | −7 | | 1 | −2 |
| 35 | | −7 | | −2 | −2.5 |
| 36 | | −7 | | −5 | −2.5 |
| 37 | | −7.5 | | −7 | −2.5 |
| 38 | Thaw | −8 | Thaw | −10 | −3 |
| 39 | | −8 | | −12 | −3 |
| 40 | | −8 | | −14 | −3 |
| 41 | | −8 | | −14 | −3 |
| 42 | | −8 | | −14 | −3 |
| 43 | | −8 | | −14 | −3.5 |
| 44 | | −8 | | −14 | −3.5 |
| 45 | | −7.5 | | −14 | −3.5 |
| 46 | | −7.5 | | −14 | −4 |
| 47 | | −8 | | −13 | −4 |
| 48 | | −8 | | −12 | −4 |
| 49 | | −8.5 | | −10 | −4.5 |
| 50 | | −8.5 | | −9 | −4.5 |
| 51 | | −8.5 | | −7 | −4.5 |
| 52 | | −8.5 | | −7 | −4.5 |
| 53 | | −9 | | −6 | −4.5 |
| 54 | | −9 | | −6 | −5 |
| 55 | | −9 | | −6 | −5 |
| 56 | | −9 | | −6 | −5 |
| 57 | | −9 | | −6 | −5 |
| 58 | | −9 | | −6 | −5.5 |
| 59 | | −9.5 | | −6 | −5.5 |
| 60 | | −9.5 | | −6 | −5.5 |

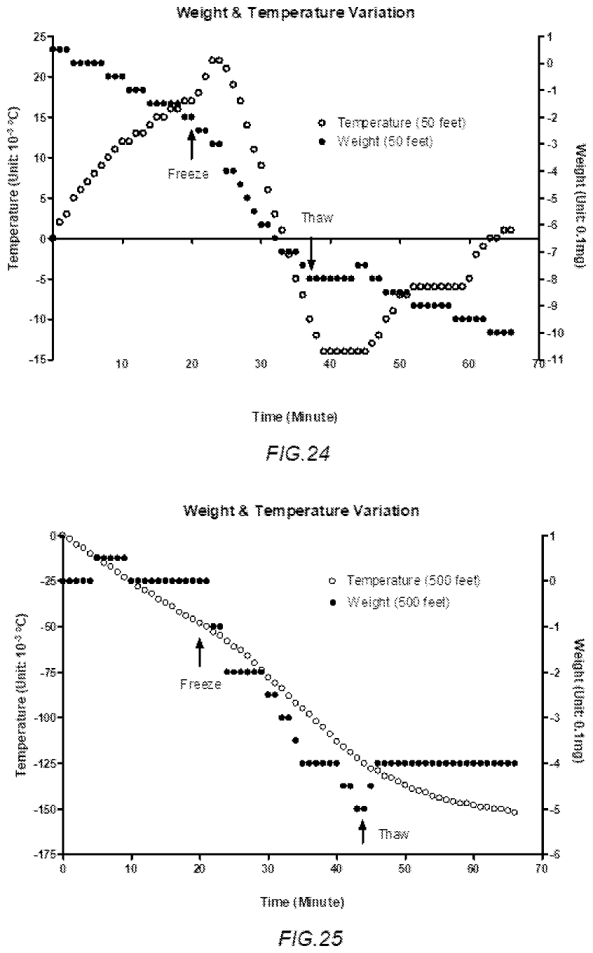

FIG. 22 is statistical chart view of several weight data sets obtained on target substance including the sample data in Column 2 and Column 3. FIG. 23 is chart view of several weight data sets obtained on target substance sample data in Column 2. FIG. 24 is chart view of data sets in Column 2 and Column 3 of Table 3. FIG. 25 is chart view of another data sets obtained in the same way as those in Column 2 and Column 3 of Table 3.

As shown in Column 2 of Table 3 and FIG. 22-25, before the submersion of said container 122 into liquid nitrogen the weight of said water 111a in said container 112a drifted lower very slowly. But almost immediately after said container 122 was submerged into liquid nitrogen, during which the temperature and physical properties of said water 121 being manipulated drastically changed, the weight of said water 111a dropped at an increased rate, and after the frozen water 121 was taken out the liquid nitrogen and thawed in warm water the weight of said water 111a first stopped dropping and, in some cases, even rose before resuming drifting lower. In Table 3 and FIG. 22-25, the weight and temperature at the starting point of measurements are set to zero.

In contrast, as shown in Column 4 of Table 3 and FIG. 22, the control experiments did not show such dynamics. The weight difference from control in which no freeze-thaw was done at the point of thawing is about 0.2 mg. Statistical analysis performed on all data collected after freezing for 10 min show that the results are significantly different under these different treatments/settings. In addition, as shown in Column 3 of Table 3 and FIG. 24-25, the dynamics of the temperature variations during the same three stages of manipulation are also very clear.

The applicant found in said first variation of the first setup that when said water 121 was quickly heated to boiling on a household gas stove instead of being frozen in liquid nitrogen, a brief rise of weight of said water 111a in the range of about 0.05 mgwere repeated observed in several experiments conducted so far.

The applicant further found in said fourth variation of the first setup that when the originating substance 121, the 250 m/water, was poured into the 200-gallon water tank instead of being frozen in liquid nitrogen, small but noticeably increased weight losses were repeatedly observed in the several experiments conducted to date. More specifically, before mixing of said water 121 with the water in the water tank the weight of said water 111a in said container 112a drifted lower very slowly, but within short time measured in minutes after said water 121 in said container 122 was poured into said water tank, during which the water in the said tank got quantum-entangled with said water 121, the weight of the water 111a in said container 112a dropped at small but increased rate for a period of time.

Columns 2, 4 and 5 in Table 4 respectively show (1) sample data on temperature change obtained in the third variation of the first set of experiment described above; (2) one sample data on the pH variation of said first substance 211, the 200 m/tap water, in the second set of experiment; and (3) one sample data on the pH variation of said first substance 211, the 200 m/tap water, in the control group of the second set of experiments:

TABLE 4

| | Treatment | | | | | |
|---|---|---|---|---|---|---|
| | Temp | | | Treatment | | Control |
| Time (Minute) | (Unit: 10⁻³° C.) | | Time (Minute) | pH (Unit: 10⁻³) | | pH (Unit: 10⁻³) |
| 1 | | 0 | 1 | | 0 | 0 |
| 2 | | 2 | 2 | | 0 | 0 |
| 3 | | 4 | 3 | | −1 | 0 |
| 4 | | 6 | 4 | | −1 | −1 |
| 5 | | 8 | 5 | | −1 | −2 |
| 6 | | 11 | 6 | | −2 | −2 |
| 7 | | 14 | 7 | | −3 | −3 |
| 8 | Add HCl | 16 | 8 | HCl Exposure | −3 | −3 |
| 9 | | 17 | 9 | | −3 | −2 |
| 10 | | 19 | 10 | | −3 | −2 |
| 11 | | 21 | 11 | | −3 | −2 |
| 12 | | 24 | 12 | | −3 | −1 |
| 13 | | 27 | 13 | | −3 | −1 |
| 14 | | 30 | 14 | | −3 | 0 |
| 15 | | 32 | 15 | | −3 | 0 |
| 16 | | 32 | 16 | | −2 | 0 |
| 17 | | 32 | 17 | | −2 | 0 |
| 18 | | 32 | 18 | | −2 | 0 |
| 19 | | 32 | 19 | | −2 | 0 |
| 20 | | 31 | 20 | | −2 | 0 |
| 21 | | 31 | 21 | | −2 | −1 |
| 22 | | 30 | 22 | | −3 | −1 |
| 23 | | 29 | 23 | | −3 | −1 |
| 24 | | 28 | 24 | | −3 | −1 |
| 25 | | 27 | 25 | | −3 | 0 |
| 26 | | 26 | 26 | | −3 | 0 |
| 27 | | 25 | 27 | | −3 | 1 |
| 28 | Add NaOH | 24 | 28 | | −3 | 0 |
| 29 | | 23 | 29 | | −3 | 0 |
| 30 | | 22 | 30 | | −3 | 0 |
| 31 | | 21 | 31 | | −4 | −1 |
| 32 | | 21 | 32 | | −4 | −1 |
| 33 | | 21 | 33 | | −5 | −2 |
| 34 | | 22 | 34 | | −5 | −2 |
| 35 | | 21 | 35 | | −6 | −2 |
| 36 | | 19 | 36 | | −6 | −2 |
| 37 | | 18 | 37 | | −6 | −2 |
| 38 | | 16 | 38 | | −8 | −2 |
| 39 | | 13 | 39 | | −8 | −2 |
| 40 | | 10 | 40 | | −8 | −2 |
| 41 | | 8 | 41 | | −10 | −2 |
| 42 | | 5 | 42 | | −10 | −3 |
| 43 | | 2 | 43 | | −11 | −3 |
| 44 | | −1 | 44 | | −11 | −3 |
| 45 | | −4 | 45 | | −11 | −3 |
| 46 | | −8 | 46 | | −12 | −3 |
| 47 | | −11 | 47 | | −12 | −3 |
| 48 | Heat | −14 | 48 | | −12 | −3 |
| 49 | | −17 | 49 | | −13 | −3 |
| 50 | | −20 | 50 | | −13 | −3 |
| 51 | | −22 | 51 | | −13 | −3 |
| 52 | | −23 | 52 | | −13 | −3 |
| 53 | | −24 | 53 | | −13 | −3 |
| 54 | | −23 | 54 | | −13 | −3 |
| 55 | | −26 | 55 | | −13 | −3 |
| 56 | | −29 | 56 | | −14 | −3 |
| 57 | | −32 | 57 | | −14 | −3 |
| 58 | | −35 | 58 | | −14 | −3 |
| 59 | | −38 | 59 | | −14 | −3 |
| 60 | | −41 | 60 | | −14 | −3 |

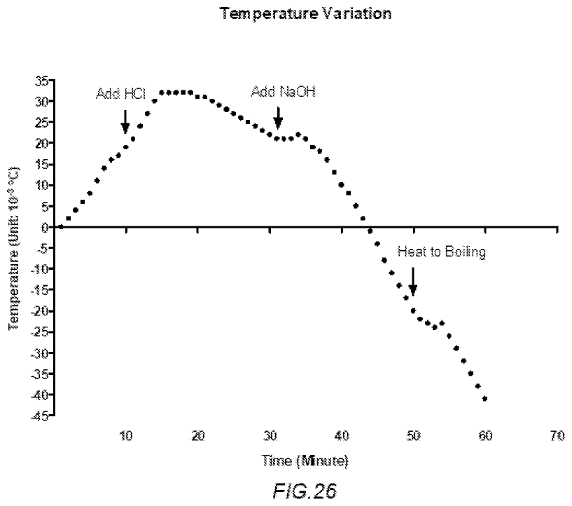

As shown in Column 2 of Table 4, and FIG. 26 which is chart view of Column 2 of Table 4, the signatures of the manipulation induced temperature changes in said third variation of the first setup were clear. The applicant further found that when said 5 m/HCl was added to the water 121, the pH value in the water being measured decreased. In Column 2 of Table 4 and FIG. 26, the general background trend of the temperature first increasing, flattening and decreasing was due to environmental temperature change.

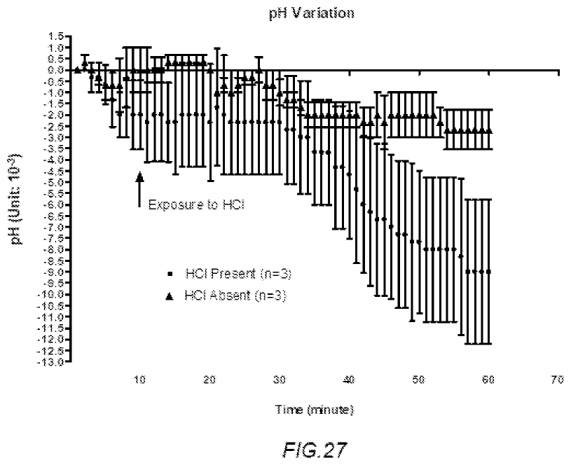

As shown in Column 4 of Table 4, and FIG. 27 which is statistical chart view of several data sets, in about 30 min after the second container 222 containing said second substance 221, the 100 m/concentrated HCl (38% by weight), was disposed behind the first container 212 at the said distance of 500 cm and on the path of said laser light, during which the water 211 in the first container 212 got quantum-entangled with the HCl 221 in the second container 222, the pH value of the water 211 in the first container 212 steadily decreased. In Columns 4 & 5 of Table 4 and FIG. 27, the pH value at the starting point of measurement is set to zero.

In contrast, as shown in Column 5 of Table 4 and FIG. 27, the control experiment did not show such dynamics. Also, the said laser did not affect the temperature of the water in the first reservoir significantly during the whole treatment. The difference in pH value from control in which HCl was absence is about 0.070 after 50 min of exposure to HCl. Statistical analysis performed on all data collected after exposure to HCl for 30 min show that the results are significantly different from control.

With all experimental setups and their variations described herein, the applicant has observed clear and reproducible non-local effects with the aids of high-precision analytical instruments and under well-controlled conditions. The physical observables used for measuring the non-local effects are simple ones which can be measured with high precisions. These non-local effects are, even under the most stringent statistical analysis, significantly above and beyond what were noticeable in the control experiments.

Through careful analysis, I have excluded the possibility that the observed weight variation was a secondary local effect due to heat loss and/or sensitivity drift of balance associated with temperature change induced by the manipulation of said originating substance. First, during the period of said manipulation the total temperature change caused by said manipulation was less than 0.08° C. so the total heat loss for the 175 m/water 111a in the container 112a was about 60J. In contrast, the weight loss during remote manipulation was on average about 0.25 mgwhich is 22.5×10⁹J in energy unit. Second, said container 112a and the pan of the analytic balance 114a were separated by a 1-inch white foam to prevent heat transfer to said analytic balance 114a. Even in the highly unlikely scenario that this temperature change somehow affected the overall temperature of the analytical balance 114a, the associated sensitivity drift of the balance was about 0.03 mg which is 10 times smaller than what's actually observed. Therefore, the observed gravity variation is a genuine and direct non-local effect associated with quantum entanglement.

The applicant chose to use liquid nitrogen in said 25-liter Dewar placed at said distant locations for manipulating said originating substance 121 in said experiments because said liquid nitrogen provided drastic changes in the temperature and properties of the water 121 in a very short period of time. The applicant's expectation was that, if the quantum entities inside the water 111 and 111a being measured were able to sense the changes experienced by the quantum entities in the water 121 being manipulated through quantum entanglement and further utilize the information associated with the said changes, the chemical, thermal and gravitational properties of the water 111 and 111a might be affected through quantum entanglement mediated non-local processes (Hu, H. P., & Wu, M. X. Photon induced non-local effect of general anesthetics on the brain. NeuroQuantology 4, 17-31 (2006); Hu, H. P., & Wu, M. X. Non-local effects of chemical substances on the brain produced through quantum entanglement. Progress in Physics v3, 20-26 (2006)). While the applicant does not wish to be bound by a particular suggested mechanism, the most logical explanation for all the observed non-local effects is that they are the consequences of non-local processes mediated by quantum entanglement between quantum entities in the water being measured and the remote water being manipulated as more specifically illustrated below.

First, when the pH value of the water 121 in the container 122 is high or low or is changing under said manipulations such as said cooling, heating or addition of said acidic or alkaline chemical, the pH value of said water 111 being measured shift in the same direction as that of said water 121 under the non-local influence of said water 121 in said container 122 mediated through quantum entanglement between said water 111 and 121 and, under the condition that said container 112 is able to exchange energy with its local environment, as if H+in said water 121 is directly available to said water 111 in said container 112.

Second, when temperature of the water 121 in the container 122 is extremely low or high or is changing under said manipulations such as said cooling, heating or addition of heat-generating chemicals such as concentrated HCl or NaOH powder, the temperature of said water 111 being measured shift in the same direction as that of said water 121 under the non-local influence of said water 121 in said container 122 mediated through quantum entanglement between said water 111 and 121 and, under the condition that said container 112 is able to exchange energy with its local environment so that the local thermodynamic energy is conserved, as if the heat or lack of it in said container 122 is directly available to said water 111 in said container 112.

Third, when the water 121 in the container 122 is manipulated though cooling, heating or mixing with large quantum-entangled mass such that the quantum entanglement of the water 121 under manipulation with its local environment changes, the weight of the water 111a in said container 112a also changes under the non-local influence of said manipulation mediated through quantum entanglement between said water 111a being weighed and 121 being manipulated.

While the applicant does not wish to be bound by any particular quantum entities suggested herein, it is believed that nuclear spins and/or electron spins respectively inside the target substance and originating substance are the quantum entities responsible for mediating the observed non-local effects since nuclear spins and electron spins are the natural targets of interactions for reasons discussed below.

Water contains vast numbers of nuclear spins carried by ¹H. These spins form complex intra- and inter-molecular networks through various intra-molecular J- and dipolar couplings and both short- and long-range intermolecular dipolar couplings. Further, nuclear spins have relatively long relaxation times after excitations. Thus, when a nematic liquid crystal is irradiated with multi-frequency pulse magnetic fields, its ¹H spins can form long-lived intra-molecular quantum coherence with entanglement for information storage (Khitrin, A. K., Ermakov, V. L. & Fung, B. M. Information storage using a cluster of dipolar-coupled spins. Chem. Phys. Lett. 360, 161-166 (2002)). Long-lived (˜0.05 ms) entanglement of two macroscopic electron spin ensembles in room temperature has also been achieved (Julsgaard, B., Kozhekin, A. & Polzik, E. S. Experimentally long-lived entanglement of two macroscopic objects. Nature 413, 400-403 (2001)). Furthermore, spin is a fundamental quantum process and was shown to be responsible for the quantum effects in both Hestenes and Bohmian quantum mechanics (Hu, H. & Wu, M. Spin as primordial self-referential process driving quantum mechanics, spacetime dynamics and consciousness. NeuroQuantology 2:41-49 (2004)). Thus, we suggest that quantum-entangled nuclear spins and/or electron spins are likely the mediators of all observed non-local effects reported here (Hu, H. P., & Wu, M. X. Photon induced non-local effect of general anesthetics on the brain. NeuroQuantology 4, 17-31 (2006); Hu, H. P., & Wu, M. X. Non-local effects of chemical substances on the brain produced through quantum entanglement. Progress in Physics v3, 20-26 (2006)).

In short, through the above described experiments, I have discovered that (1) the pH value of water in a detecting reservoir quantum-entangled with water in a remote reservoir changes in the same direction as that in the remote water when the latter is manipulated under the condition that the water in the detecting reservoir is able to exchange energy with its local environment; (2) the temperature of water in a detecting reservoir quantum-entangled with water in a remote reservoir can change against the temperature of its local environment when the latter is manipulated under the condition that the water in the detecting reservoir is able to exchange energy with its local environment; and (3) the gravity of water in a detecting reservoir quantum-entangled with water in a remote reservoir can change against the gravity of its local environment when the latter was remotely manipulated; Thus, among other things I have realized non-local signaling using three different physical observables—pH value, temperature and gravity.

My invention and discovery also make it clear that (1) the properties of all matters can be affected non-locally through quantum entanglement mediated processes; (2) physically, chemically and/or biologically meaningful information can be transmitted from one system or location to the other through quantum entanglement; (3) quantum entanglement can be used to deliver the therapeutic effects of many drugs to biological systems such as human bodies without ever physically administrating the said drugs to the said systems; (4) quantum entanglement alone can be used for communications of both quantum and classical information; (5) many substances of nutritional and even recreational values can be repeatedly administrated to the human body through the said technologies; and (6) quantum entanglement can also be used to entangle two or more biological objects such as human brains for legitimate purposes.

It will be evident from the above that there are other embodiments which are clearly within the scope and spirit of the present invention, although they were not expressly set forth above. Therefore, the above disclosure is exemplary only, and the actual scope of my invention is to be determined by the claims.

What is claimed is:

1. A method of producing a non-local effect in a target substance through manipulating an originating substance and detecting said non-local effect which comprises the steps of:
selecting a substance which comprises said target substance and said originating substance;
generating a plurality of quantum entanglements within a plurality of quantum entities in said substance by irradiating said substance with magnetic pulse, laser light or microwave, or letting said substance sit for at least thirty days;
separating said substance into said target substance and said originating substance;
positioning said target substance at a first location in a first stable environment and said originating substance at a second location in a second stable environment;
cooling, heating or adding a third substance to said originating substance; and
detecting with a high-precision instrument a change in weight, temperature and/or pH value of said target substance;
whereby said non-local effect is produced through a non-local process mediated by said quantum entanglements and detected, said non-local effect being said change in weight, temperature and/or pH value of said target substance.

2. A method as in claim 1 for non-locally transmitting a message from said second location to said first location wherein said positioning step further comprises encoding said message to be carried by said non-local effect; and said detecting step further comprises decoding said message at said first location.

3. A method as in claim 1 wherein said substance comprises water, oil, water-based medium or oil-based medium.

4. A method as in claim 2 wherein said substance comprises water, oil, water-based medium or oil-based medium.

5. A method of producing a non-local effect in a target substance through manipulating an originating substance and detecting said non-local effect which comprises the steps of:
selecting said target substance and said originating substance, a first plurality of quantum entities in said target substance being in a plurality of quantum entanglements with a second plurality of quantum entities in said originating substance;
positioning said target substance at a first location in a first stable environment and said originating substance at a second location in a second stable environment;
cooling, heating or adding a third substance to said originating substance; and
detecting with a high-precision instrument a change in weight, temperature and/or pH value of said target substance;
whereby said non-local effect is produced through a non-local process mediated by said quantum entanglements and detected, said non-local effect being said change in weight, temperature and/or pH value of said target substance.

6. A method as in claim 5 for non-locally transmitting a message from said second location to said first location wherein said positioning step further comprises encoding said message to be carried by said non-local effect; and said detecting step further comprises decoding said message at said first location.

7. A method as in claim 5 wherein said target substance comprises a first volume of water, oil, water-based medium or oil-based medium; and said originating substance comprises a second volume of water, oil, water-based medium or oil-based medium.

8. A method as in claim 6 wherein said target substance comprises a first volume of water, oil, water-based medium or oil-based medium; and said originating substance comprises a second volume of water, oil, water-based medium or oil-based medium.

9. A method of producing a non-local effect in a target substance through manipulating an originating substance and detecting said non-local effect which comprises the steps of:
selecting said target substance and said originating substance;
generating a plurality of quantum entanglements between a first plurality of quantum entities in said target substance and a second plurality of quantum entities in said originating substance by contacting said target substance with said originating substance for at least 30 days and separating said target substance and said originating substance, or disposing said target substance next to said originating substance and irradiating said target substance and said originating substance with magnetic pulse, laser light or microwave;
positioning said target substance at a first location in a first stable environment and said originating substance at a second location in a second stable environment;
cooling, heating or adding a third substance to said originating substance; and
detecting with a high-precision instrument a change in weight, temperature and/or pH value of said target substance;
whereby said non-local effect is produced through a non-local process mediated by said quantum entanglements and detected, said non-local effect being said change in weight, temperature and/or pH value of said target substance.

10. A method as in claim 9 for non-locally transmitting a message from said second location to said first location wherein said positioning step further comprises encoding said message to be carried by said non-local effect; and said detecting step further comprises decoding said message at said first location.

11. A method as in claim 9 wherein said target substance comprises a first volume of water, oil, water-based medium or oil-based medium; and said originating substance comprises a second volume of water, oil, water-based medium or oil-based medium.

12. A method as in claim 10 wherein said target substance comprises a first volume of water, oil, water-based medium or oil-based medium; and said originating substance comprises a second volume of water, oil, water-based medium or oil-based medium.
